(19) United States
(12) Patent Application Publication
(10) Pub. No.: US 2014/0235568 A1
(43) Pub. Date: Aug. 21, 2014

(54) GEMCITABINE AMIDE DERIVATIVE AND PREPARATION METHOD AND USE THEREOF

(71) Applicants: Yunlong Song, Shanghai (CN); Zhiyu Shao, Shanghai (CN)

(72) Inventors: Yunlong Song, Shanghai (CN); Zhiyu Shao, Shanghai (CN)

(21) Appl. No.: 14/347,504

(22) PCT Filed: Sep. 26, 2012
(86) PCT No.: PCT/CN2012/082090

§ 371 Date: Mar. 26, 2014

(30) Foreign Application Priority Data
Sep. 26, 2011 (CN) 201110288814.6

(51) Int. Cl. C07H 19/06; C07H 1/00

(52) U.S. Cl. CPC C07H 19/06; C07H 1/00
USPC 514/49; 536/28.51

(57) ABSTRACT

The present invention relates to the field of medical technology, and in particular relates to a kind of gemcitabine amide derivative with a novel structure. The new compounds of the present invention are very active with regard to many tumour cells such as human lung cancer, colon cancer, breast cancer and liver cancer etc., and therefore can be used for preparing anti-tumour drugs. In addition, these compounds also have anti-viral activity. Also disclosed are a preparation method for the compounds, a pharmaceutical composition containing the compounds and the use thereof in preparing drugs against tumours and viruses etc.

### TECHNICAL FIELD

The present invention relates to the field of medical technology, more specifically, to a gemcitabine amide derivative. The present invention also relates to a composition of the compound, preparation method and use of the compound in the preparation of antitumor and antiviral medicaments.

### BACKGROUND ART

Gemcitabine hydrochloride (2′,2′-difluoro-2′-deoxycytidine hydrochloride, with trade name Gemzar) was a marketed antineoplastic drug of Eli Lilly and Company, USA. The medicinal form is freeze-dried powder formulation, which was approved in the treatment of pancreatic cancer, breast cancer and non-small cell lung cancer. In addition, gemcitabine hydrochloride has antiviral activity. Studies have reported that gemcitabine can be used in the treatment of flavivirus infection including hepatitis C virus. The patient's body hepatitis C viral load can be rapidly reduced with 2 logarithmic unit or more within a few days (1-2 days in some cases) at the minimum dose (Chinese invention patent, application number: 03808085,X). The oral bioavailability of gemcitabine was rather poor, therefore, the clinical drug administration is an intravenous infusion with a dose from about 1000 to about 1250 mg/m² in 30 minutes, once a week, administered for up to seven weeks, followed by a rest period of a week without the treatment.

Studies have shown that the poor oral bioavailability of gemcitabine is due to the presence of first-pass metabolism. Besides, oral administration will lead to dose-limiting intestinal damage. To overcome the first-pass effect of gemcitabine, and develop novel gemcitabine derivatives that can be administered orally, gemcitabine prodrug was frequently studied. Among them, the amide derivatives of gemcitabine were ever considered to be useful intermediates for the structural modification of gemcitabine. Researchers in Eli Lilly and Company found that N4-valproyl gemcitabine (LY-2334737) may be administered orally, and maintains superior stability in the gastrointestinal tract and lower toxicity than oral gemcitabine, while provides comparable therapeutic effect by low-dose oral administration with intravenous gemcitabine. The following studies demonstrated that LY-2334737 was primarily hydrolyzed into gemcitabine by carboxylesterase 2 in human body, which also indicates that its efficacy is affected by expression level of carboxylesterase 2.

The present invention provides a gemcitabine amide derivative with new structure and superior activity. They can be injected or orally administered. This provided the basis for the development of new gemcitabine-based antitumor drug.

### SUMMARY OF INVENTION

The object of the present invention is to provide a new gemcitabine amide derivative, or its pharmaceutically acceptable salt, solvate or polymorph thereof. The present invention also discloses the preparation method, medical use and composition of the compound.

In the first aspect of the invention, a gemcitabine amide derivative of the general formula (I), or the pharmaceutically acceptable salt, solvate or polymorph thereof is provided.

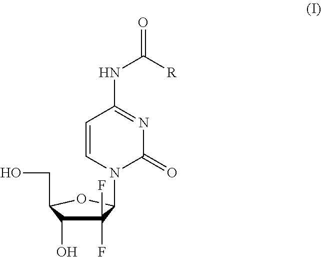

wherein, R is a substituted or non-substituted C1-21 straight-chain or branched alkyl, with the proviso that n-butyl, 1-propylbutyl, n-undecyl and n-heptadecyl are excluded; R can also be C1-8 alkyl directly connected with the substituted phenyl;
the “substituted” refers to being substituted by one or more of the following substituents: hydrogen, C1-6 alkyl, C1-6 haloalkyl, C2-5 alkenyl, C2-5 alkynyl, C1-5 alkyloxy, halogen, nitro, cyano, hydroxyl, amino, carboxyl, and oxo.

In another preferred embodiment, R is a straight-chain alkyl;

In another preferred embodiment, R is any one of the n-propyl, n-heptyl or phenylpropyl.

The present invention also provides a use of above compound of the invention in the preparation of antitumor or antiviral agents. Preferably, the antiviral agent is anti-hepatitis C virus agent; or the tumor is susceptible tumor.

The present invention also provides a use of a gemcitabine amide derivative of the general formula (I) in the preparation of antitumor or antiviral agents, wherein, R is n-butyl.

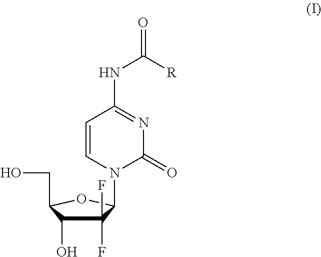

Preferably, the antiviral agent is anti-hepatitis C virus agent; or the tumor is susceptible tumor.

The present invention also provides a composition comprising a safe and effective amount of the above compound and a pharmaceutically acceptable carrier.

In addition, the present invention also provides a composition comprising a safe and effective amount of gemcitabine amide derivative of the general formula (I) and a pharmaceutically acceptable carrier, wherein, R is n-butyl;

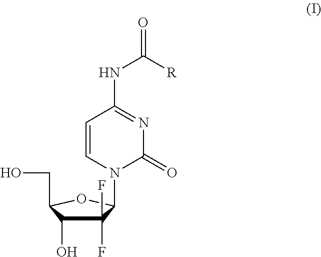

Preferably, the composition is a pharmaceutical composition, including (but not limited to) a tablet, capsule or injection; More preferably, the composition is enteric-coated formulations (enteric-coated tablet or enteric-coated capsules).

In the embodiment of the present invention includes, but are not limited to the amide derivatives of gemcitabine shown below:

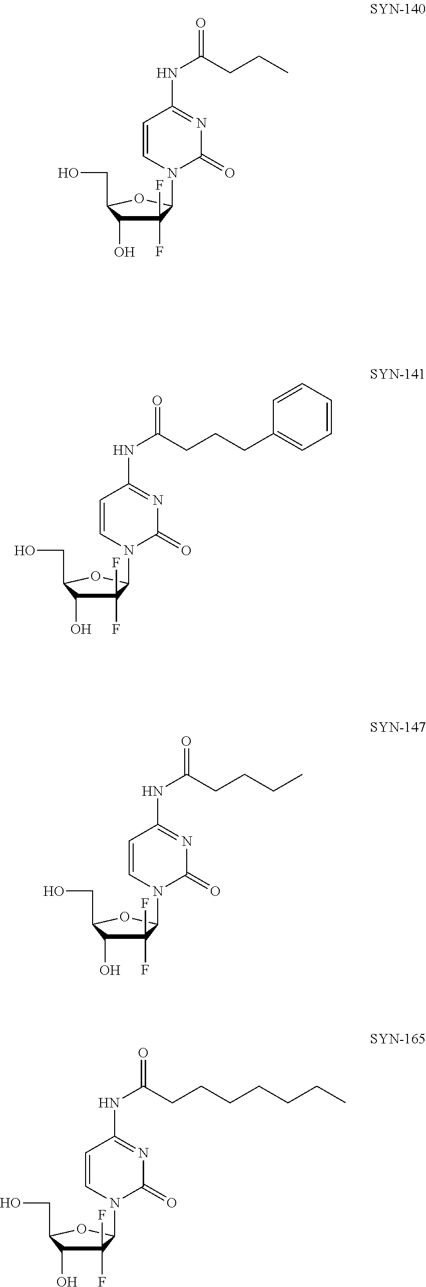

In addition, the present invention also includes the compound of any one of the following general formula with n=1-20. More specifically, when n=7, 8, 14, 18, 20, the labels for the corresponding compound refers to the particular embodiments.

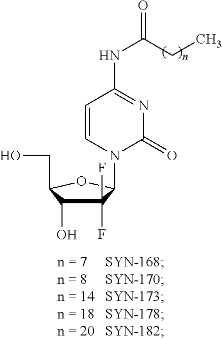

The another object of the present invention is to provide a preparation method of the mentioned gemcitabine amide derivative, which comprises the following steps: the mixture of gemcitabine, anhydrous pyridine and triethylchlorosilane, was stirred at room temperature; in the meantime, corresponding carboxylic acid was dissolved in acetonitrile, then the condensing agent was added and stirred at room temperature, this solution was added dropwise to the forementioned mixture of gemcitabine, stirred overnight while maintaining at 30-60° C., the reaction mixture was concentrated, and the residue was dissolved in an appropriate amount of an organic solvent, trifluoroacetic acid was added dropwise and stirred to recover the solvent and give the crude product, which was purified by silica gel column chromatography to provide the pure target product. The “condensing agent” may be independently selected from the group consisting of: N,N′-carbonyldiimidazole (CDI), triphenylphosphine, 1-hydroxy-benzotriazole (HOBT), N-hydroxy-7-azabenzotriazole (HOAT), 1-ethyl-3-(3-dimethylaminopropyl) carbodiimide hydrochloride (EDCI), (benzotriazol-1-yl-oxytripyrrolidinophosphonium hexafluorophosphate (PyBOP), 4-dimethylaminopyridine (DMAP) or dicyclohexyl carbodiimide (DCC). Preferably, the condensing agent is N,N′-carbonyldiimidazole.
wherein, the carboxylic acid is RCOOH, and R is as defined as previous.

Another object of the present invention is to provide a preparation method of the compound of formula (I) of the present invention, comprising the steps: a condensation reaction of gemcitabine with RCOOH is performed in an inert solvent to form a compound of general formula (I);
wherein R in each formula is defined as above.

In another preferred embodiment, the mentioned gemcitabine is a hydroxyl-protected gemcitabine.

In another preferred embodiment, further step is also comprised after the condensation reaction: a compound of general formula (I) is formed by removal of the protective group of the condensation products.

### BRIEF DESCRIPTION OF THE DRAWS

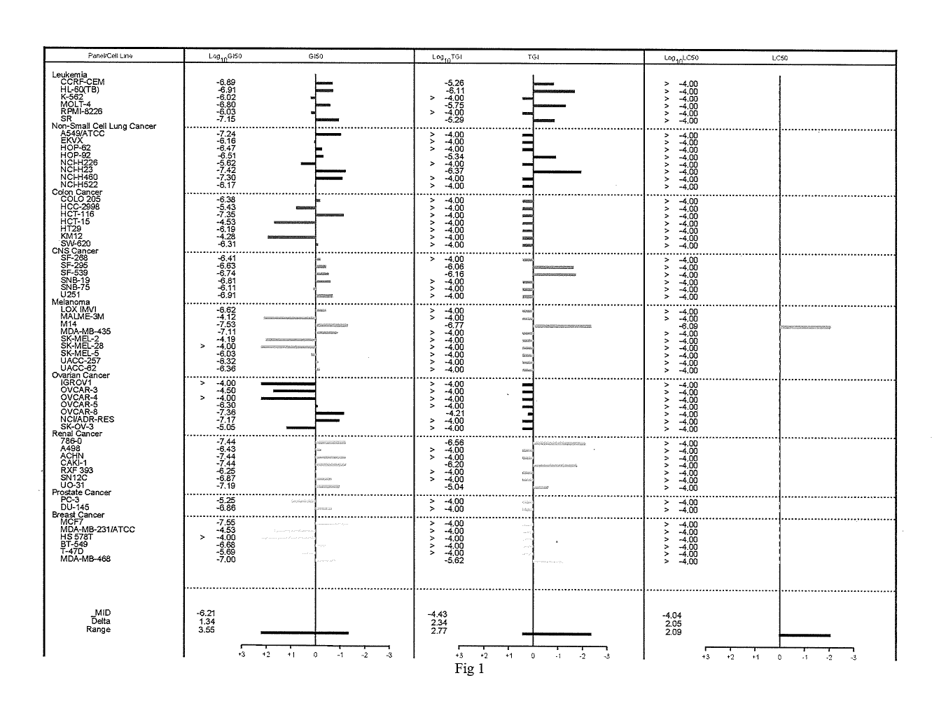

FIG. 1 is in vitro cytotoxic activity data of compound SYN-141 against NCI human 60 kinds of tumor cells.

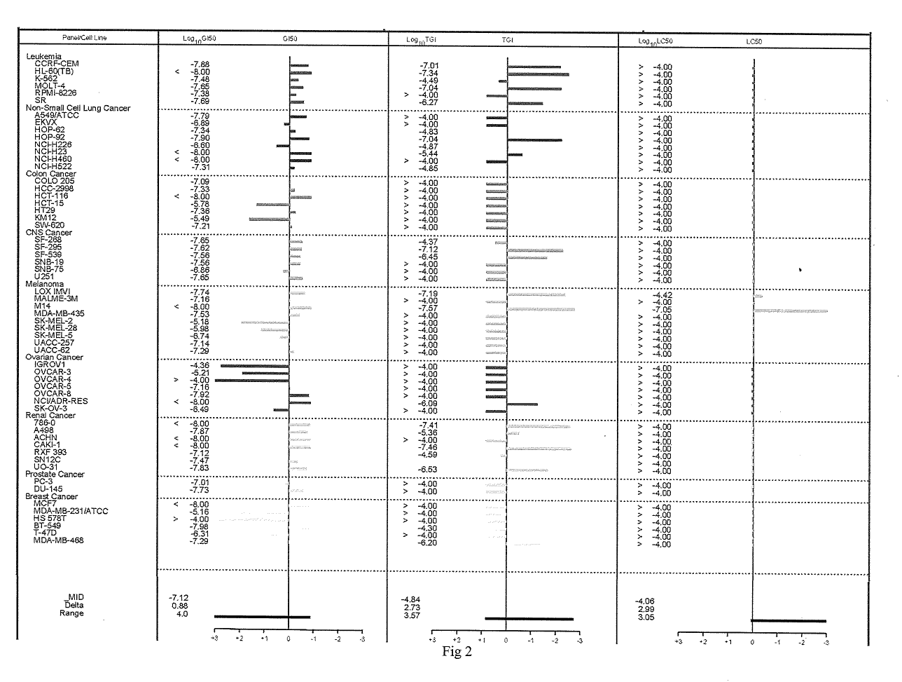

FIG. 2 is in vitro cytotoxic activity data of compound SYN-165 against NCI human 60 kinds of tumor cells.

In the figures, the unit of GI50, TGI and LC50 is M.

### DETAILED DESCRIPTION OF THE INVENTION

Based on gemcitabine which is widely used in clinic currently, the inventors designed and synthesized a class of novel structure of the gemcitabine amide derivative through extensive and in-depth research. The experimental results show that the compounds have significant antitumor activities, especially in a variety of solid tumors such as liver cancer, lung cancer, breast cancer, colon cancer. Based on these results, the present invention was completed.

The “susceptible tumor” refers to that an abnormal growth of the mammalian tissues which could be treated through injection or oral administration of general formula (I) compound. These compounds could be hydrolysed into gemcitabine in vivo, and gemcitabine have a good antitumor activity against a variety of tumor cells. Therefore, these compounds as shown in the general formula (I) are expected to be potent against many types of tumors (including solid tumors and non-solid tumors). Likewise, the compounds of general formula (I) also have antiviral activity (including flavivirus such as hepatitis C virus), because gemcitabine has antiviral activity.

Preferably, the “susceptible tumor” includes breast cancer, lung cancer, liver cancer, colon cancer, pancreatic cancer, T-cell lymphoma, soft tissue sarcoma, Hodgkin's lymphoma, non-Hodgkin's lymphoma, ovarian cancer or bladder cancer.

The “safe and effective amount” refers to that the amount of the compound that is sufficient to improve the condition, but will not cause serious side effects. The safe and effective amount is determined according to patient's age, condition, and course of treatment. The pharmaceutical composition usually contains 1-2000 mg of the present invented compound/dose, preferably 10-200 mg. Preferably, the “one dose” is an ampoule, capsule or tablet.

Gemcitabine amide derivatives in the present invention can be modified to the form of their pharmaceutically acceptable salts according to conventional methods. The salts include inorganic acid salts and organic acid salts. Inorganic acids include (but are not limited to) hydrochloric acid, sulfuric acid, phosphoric acid, diphosphonic acid, hydrobromic acid, nitric acid, etc.; organic acids include (but are not limited to) the acetic acid, maleic acid, fumaric acid, tartaric acid, succinic acid, lactic acid, p-toluenesulfonic acid, salicylic acid, oxalic acid, etc..

The compounds of the present invention have significant antitumor activities. They can be used for the treatment of tumors, which occur in esophagus, stomach, intestines, rectum, mouth, pharynx, larynx, lung, colon, breast, uterus, endometrium, ovary, prostate, testicle, bladder, kidney, liver, pancreas, bone, connective tissue, skin, eye, brain and central nervous system, etc. as well as thyroid cancer, leukemia, Hodgkin's disease, lymphoma, myeloma and the like.

The pharmacological activities of the gemcitabine amide derivatives of the present invention make them be used in the preparation of antiviral or antitumor medicaments. Therefore, the present invention further comprises the pharmaceutical compositions of these compounds or their pharmaceutically acceptable salts thereof as active ingredients. The pharmaceutical composition also contains a pharmaceutically acceptable carrier, which may be a solid form or liquid form, wherein the pharmaceutical dosage form may be tablets, capsules, powders, granules, suspensions or injections.

“Pharmaceutically acceptable carrier” means one or more compatible solid or liquid fillers or gel materials, which are suitable for human, and must have sufficient purity and sufficiently low toxicity. “Compatibility” herein means that the components of the composition can be blended with the compounds of the invention or with each other, and would not significantly reduce the efficacy of the compounds. Some examples of pharmaceutically acceptable carriers include cellulose and the derivatives thereof (such as sodium carboxymethyl cellulose, sodium ethyl cellulose, cellulose acetate, etc.), gelatin, talc, solid lubricants (such as sodium stearate, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween), wetting agent (such as sodium dodecyl sulfate), coloring agents, flavoring agents, stabilizers, antioxidants, preservatives, pyrogen-free water, etc..

Using the pharmaceutical composition means a safe and effective amount of a compound of the present invention is used to mammals (such as human) that are in need of such treatments, wherein the dose in administration is a pharmaceutically effective dose. For a human with 60 kg body weight, the dose is usually 1˜2000 mg, preferably 20 to 500 mg. Of course, the specific dose should also consider the route of administration, the patient's health status and other factors, which are under a skilled physician's control.

The main advantages of the present invention, in that:
(A) It provides a series of gemcitabine amide derivatives with novel structures.
(B) They have broad-spectrum antitumor activities, significantly inhibite the growth of a variety of solid tumors.
(C) They have stable chemical structures, with the hope to developped into innovative anticancer drugs.

### DESCRIPTION OF THE PREFERRED EMBODIMENTS

The present invention will be further illustrated below with reference to specific examples. It should be understood that these examples are only to illustrate the present invention but not to limit the scope of the present invention. The experimental methods with no specific conditions described in the following examples are generally performed under conventional conditions or according to the manufacture's instruction. Unless indicated otherwise, the percentages, or parts are calculated by weight. All starting materials and reagents are well known in prior art and can be readily obtained or be prepared using the method described in the literature.

### Example 1

### Synthesis of N⁴-n-butyryl gemcitabine (SYN-140)

In a 50 mL round-bottomed flask were successively added gemcitabine (0.44 g, 1.67 mmol), anhydrous pyridine (5 mL), and triethylchlorosilane (1.1 mL). The mixture was stirred for 1.5 hours at room temperature, which is solution A. In the meantime, n-butyric acid (0.16 g, 1.81 mmol) was dissolved in acetonitrile (4 mL), followed by the addition of carbonyl diimidazole (0.33 g, 2.03 mmol). The mixture was stirred at room temperature for 0.5 hours. This solution was added dropwise to the solution A, and stirred at 60° C. overnight. The reaction solution was evaporated to remove solvent under reduced pressure, and the residue was dissolved in methanol (5 mL). Then trifluoroacetic acid (1 mL) was added dropwise, stirred for 0.5 hours. The reaction mixture was poured into ethyl acetate (50 mL), the solid precipitate was collected, and the solution was washed with saturated brine (15 mL×2) and water (15 mL). The organic phase was dried over anhydrous sodium sulfate. The solvent was recovered under reduced pressure, and the residue and the precipitated solids were combined to yield crude product. The crude product was purified by column chromatography (SiO₂, methanol-chloroform from 2:98 to 4:96). The purity of the product was higher than 95%, yield: 85%.

Mp 198° C., ¹H NMR (DMSO-d6) δ:11.0 (1H, s), 8.22 (1H, d, J=7.8 Hz), 7.28 (1H, d, J=7.8 Hz), 6.29 (1H, d, J=6.6 Hz), 6.16 (1H, t, J=7.2 Hz), 5.26 (1H, brs), 4.18 (1H, m), 3.88 (1H, m), 3.79 (1H, m), 3.64 (1H, m), 2.37 (2H, t, J=7.2 Hz), 1.56 (2H, m), 0.87 (3H, t, J=7.2 Hz). ESIMS m/z (rel intensity): 334 (M+H⁺, 100).

### Example 2

### Synthesis of N⁴-(4-phenyl)butyryl Gemcitabine (SYN-141)

Prepared by the method in Example 1, except that 4-phenyl butyric acid (1.81 mmol) replaced n-butyric acid.

Mp 111° C., ¹H NMR (DMSO-d6) δ: 11.0 (1H, s), 8.22 (1H, d, J=7.8 Hz), 7.27 (3H, m), 7.18 (3H, m), 6.29 (1H, d, J=6.6 Hz), 6.16 (1H, t, J=7.2 Hz), 5.28 (1H, brs), 4.18 (1H, m), 3.88 (1H, m), 3.80 (1H; m) , 3.65 (1H, m), 2.58 (2H, t, J=7.2 Hz), 2.45 (2H, m), 1.87 (2H, m. J=7.2 Hz), ESIMS m/z (rel intensity): 410 (M+H⁺, 100).

### Example 3

### Synthesis of N⁴-n-valeryl Gemcitabine Gemcitabine (SYN-147)

Prepared by the method of Example 1, except that n-valeric acid (1.81 mmol) replaced n-butyric acid,

Mp 184° C., ¹H NMR (DMSO-d6) δ: 10.96 (1H, s), 8.22 (1H, d, J=7.8 Hz), 7.27 (1H, d, J=7.8 Hz), 6.29 (1H, brs), 6.16 (1H, t, J=7.2 Hz), 5.28 (1H, brs), 4.18 (1H, m), 3.88 (OH, m), 3.79 (1H, m), 3.63 (1H, m), 2.40 (2H, t, J=7.2 Hz), 1.52 (2H, m), 1.28 (2H, m), 0.86 (3H, t, J=7.2 Hz), ESIMS m/z (rel intensity): 348 (M+H⁺, 100).

### Example 4

### Synthesis of N⁴-n-octanoyl Gemcitabine (SYN-165)

Prepared by the method of Example 1, except that n-octanoic acid (1.81 mmol) replaced n-butyric acid,

Mp 139° C., 1H NMR (DMSO-d6) δ: 10.96 (1H, s), 8.22 (1H, d, J=7.5 Hz), 7.27 (1H, d, J=7.5Hz), 6.30 (1H, d, J=6.6 Hz), 6.16 (1H, t, J=7.5 Hz), 5.28 (1H, t, J=5.4 Hz), 4.18 (1H, m), 3.88 (1H, m), 3.79 (1H, m), 3.63 (1H, m), 2.39 (2H, t, J=7.2 Hz), 1.53 (2H, m), 1.24 (8H, brs), 0.84 (3H, t, J=7.2 Hz), ESIMS m/z (rel intensity): 390 (M+H⁺, 100).

### Example 5

### Synthesis of N⁴-n-nonanoyl Gemcitabine (SYN-168)

Prepared by method of Example 1, except that instead of n-nonanoic acid (1.81 mmol) of n-butyric acid,

¹H NMR (DMSO-d6) δ: 10.97 (1H, s), 8.23 (1H, d, J=7.5 Hz), 7.27 (1H, d, J=7.5 Hz), 6.30 (1H, d, J=6.6 Hz), 6.16 (1H, t, J=7.5 Hz), 5.28 (1H, t, J=5.4 Hz), 4.18 (1H, m), 3.88 (1H, m), 3.79 (1H, m), 3.63 (1H, m), 2.39 (2H, t, J=7.2 Hz), 1.53 (2H, m), 1.24 (10H, brs), 0.84 (3H, t, J=7.2 Hz)

ESIMS m/z (rel intensity): 404 (M+H⁺, 100).

### Example 6

### Synthesis of N⁴-n-decanoyl Gemcitabine (SYN-170)

Prepared by the method of Example 1, except that n-decanoic acid (1.81 mmol) replaced n-butyric acid,

¹H NMR (DMSO-d6) δ: 10.96 (1H, s), 8.22 (1H, d, J=7.5 Hz), 7.26 (1H, d, J=7.5 Hz), 6.29 (1H, d, J=6.6 Hz), 6.15 (1H, t, J=7.5 Hz), 5.28 (1H, t, J=5.4 Hz), 4.18 (1H, m), 3.88 (1H, m), 3.79 (1H, m), 3.63 (1H, m), 2.39 (2H, t, J=7.2 Hz), 1.53 (2H, m), 1.25 (12H, brs), 0.84 (3H, t. J=7.2 Hz)

ESIMS m/z (rel intensity): 418 (M+H⁺, 100).

### Example 7

### Synthesis of N⁴-palmitoyl Gemcitabine (SYN-173)

Prepared by the method of Example 1, except that palmitic acid (1.81 mmol) replaced n-butyric acid.

¹H NMR (DMSO-d6) δ: 10.97 (1H, s), 8.23 (1H, d, J=7.6 Hz), 7.27 (1H, d, J=7.6 Hz), 6.31 (1H, d, J=6.6 Hz), 6.16 (1H, t, J=7.5 Hz), 5.28 (1H, t, J=5.4 Hz), 4.17 (1H, m), 3.88 (1H, m), 3.79 (1H, m), 3.63 (1H , m), 2.38 (2H, t, J=7.2 Hz), 1.54 (2H, m), 1.24 (24H, brs), 0.84 (3H, t, J=7.2 Hz)

ESIMS m/z (rel intensity): 502 (M+H⁺, 100).

### Example 8

### Synthesis of N⁴-eicosanoyl gemcitabine (SYN-178)

Prepared by the method of Example 1, except that eicsanoic acid (1.81 mmol) repaced n-butyric acid.

¹H NMR (DMSO-d6) δ: 10.96 (1H, s), 8.22 (1H, d, J=7.5 Hz), 7.27 (1H, d, J=7.5 Hz), 6.30 (1H, d, J=6.6 Hz), 6.16 (1H, t, J=7.5 Hz), 5.28 (1H, t, J=5.4 Hz), 4.18 (1H, m), 3.88 (1H, m), 3.79 (1H, m), 3.63 (1H , m), 2.39 (2H, t, J=7.2 Hz), 1.53 (2H, m), 1.26 (32H, brs), 0.85 (3H, t, J=7.2 Hz)

ESIMS m/z (rel intensity): 558 (M+H⁺, 100).

### Example 9

### Synthesis of N⁴-n-docosanoyl gemcitabine (SYN-182)

Prepared by the method of Example 1, except that behenic acid (1.81 mmol) replaced n-butyric acid.

¹H NM (DMSO-d6) δ: 10.97 (1H, s), 8.23 (1H, d, J=7.5 Hz), 7.27 (1H, d, J=7.5 Hz), 6.30 (1H, d, J=6.6 Hz), 6.16 (1H, t, J=7.5 Hz), 5.28 (1H, t, J=5.4 Hz), 4.18 (1H, m), 3.88 (1H, m), 3.79 (1H, m), 3.63 (1H, m), 2.39 (2H, t, J=7.2 Hz), 1.53 (2H, m), 1.25 (36H, brs), 0.84 (3H, t, J=7.2 Hz)

ESIMS m/z (rel intensity): 586 (M+H⁺, 100).

### Example 10

### The Antitumor Activity Test in Vitro

1. Test Strains:

The tumor cell lines used in the experiments were as follows: A549 (human lung cancer cells), HCT116 (human colon cancer cells), HepG2 (human hepatoma cells), ZR-75-30 (human breast cancer cell), purchased from Shanghai Institute of Pharmaceutical Industry.

2. Sample Preparation:

The sample was dissloved in DMSO (Merck), and PBS was added to prepare a 1000 μg/mL of solution or a uniform suspension. And then, DMSO-containing PBS (−) was added to dilute the solution or suspension. The positive control medicaments were gemcitabine (GEM) and LY2334737 developed by Eli Lilly and Company.

3. Test Method

MTT assay: 100 μL cell suspension (4˜5×10⁴ cells/mL) was added into each well of 96-well plate, and cultured in 37° C., 5% CO₂ incubator. After 24 h, the sample solution was added in duplicate, 10 μL/well, and incubated at 37° C., 5% CO₂ for 72 h. 20 μL MTT solution (5 mg/mL) was added into each well. After 4 h, a dissolving solution was added, 100 μL/well. The plate was placed into the incubator. After dissolved, OD value at 570 nm was measured on MK-2 automatic microplate reader.

As can be seen from the test results (Table 1), the gemcitabine amide derivatives in the present invention demonsrated superior broad-spectrum antitumor activity against a variety of tumor cells, including human lung cancer, colon cancer, liver cancer and breast cancer. Table 1 shows the detailed in vitro antitumor activity data for some of the compounds.

TABLE 1
In vitro inhibition of the proliferation of human tumor cells for some compounds

| | A549 | | HCT116 | | HepG2 | | ZR-75-30 | |
|---|---|---|---|---|---|---|---|---|
| | IC50 | 100 μg/ml | IC50 | 100 μg/ml | IC50 | 100 μg/ml | IC50 | 100 μg/ml |
| Compound | (μg/ml) | IC % | (μg/ml) | IC % | (μg/ml) | IC % | (μg/ml) | IC % |
| SYN-140 | 4.26 | 64.84 | 0.395 | 87.79 | 84.38 | 50.42 | 3.01 | 89.52 |
| SYN-141 | 5.60 | 56.38 | 0.269 | 92.46 | 7.20 | 62.48 | 4.70 | 91.63 |
| SYN-147 | 7.82 | 58.09 | 0.193 | 88.14 | 0.246 | 59.49 | 1.13 | 90.22 |
| SYN-165 | 42.48 | 55.82 | 0.154 | 99.01 | 0.123 | 77.90 | 0.135 | 96.30 |
| LY-2334737 | 18.27 | 60.05 | 3.81 | 84.51 | 1.78 | 67.52 | 29.31 | 78.36 |
| GEM | 3.74 | 57.57 | 0.0237 | 78.07 | 0.263 | 53.34 | 0.454 | 96.60 |

As seen from Table 1, part of the gemcitabine amide derivatives in the present invention have shown potent, broad-spectrum antitumor activities in a variety of tumor cells including human lung cancer, colon cancer, liver cancer and breast cancer, which were significantly better than the investigational drug LY-2334737 in clinical trials, which indicates that the compounds of the present invention have good development prospects.

### Example 11

### The in Vitro Cytotoxic Activity Screening Results in the U.S. National Cancer Institute (NCI) 60 Human Tumor Cells

The cytotoxic activities of Compounds SYN-141 and SYN-165 were assayed in NCI systemactically.

As shown in FIGS. 1 and 2, the test results show that both compounds have superior, broad-spectrum antitumor activities against human leukemia, non-small cell lung cancer, colon cancer, central nervous system (CNS) cancer, melanoma, ovarian cancer, renal cancer, prostate cancer and breast cancer. The MGM of SYN-165 of is −7.12, which means the total average GI50 against 60 human tumor cells of the compound is 75.8 nM. The MGM of SYN-141 is −6.21, which means the total average GI50 against 60 human tumor cell lines of the compound is 0.62 μM.

### Example 12

### The in Vivo Antitumor Activity Test

Some of the compounds were tested in vivo using human colon cancer cells HCT116 xenografts in nude mice according to a literature method (Wei Hong, Medical Laboratory Animal Science, Sichuan Science and Technology Press, 2nd edition, Chengdu, 2001, 595-596). The compounds SYN-140, SYN-141, SYN-147 and SYN-165 have potent in vivo antitumor activity when they were dosed in 0.1-10 mg/kg body weight. They were significantly better than the positive control drug LY2334737 at the same dose.

In summary, the gemcitabine amide derivatives in the present invention have broad-spectrum antitumor activities, especially in lung cancer, colon cancer, liver cancer, and breast cancer. The invention is worth of further development. The present invention opened up new ways and directions for further research and development of new anticancer drugs.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1-11. (canceled)

12. A gemcitabine amide derivative of the general formula (I), or the pharmaceutically acceptable salt, solvate or polymorph thereof: 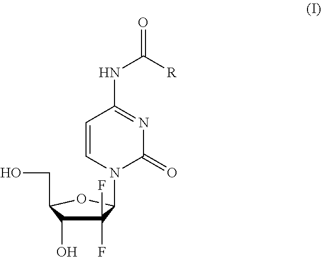
wherein, R is a substituted or non-substituted C1-21 straight-chain or branched alkyl, with the proviso that n-butyl, 1-propylbutyl, n-undecyl and n-heptadecyl are excluded; R can also be C1-8 alkyl directly connected with the substituted phenyl;
the “substituted” refers to being substituted by one or more of the following substituents: hydrogen, C1-6 alkyl, C1-6 haloalkyl, C2-5 alkenyl, C2-5 alkynyl, C1-5 alkyloxy, halogen, nitro, cyano, hydroxyl, amino, carboxyl, and oxo.

13. The compound according to claim 12, wherein, R is a straight-chain alkyl.

14. The compound according to claim 12, wherein, R is n-propyl, n-heptyl, or phenylpropyl.

15. A use of the compound of claim 12, wherein, the compound is used in the preparation of antitumor or antiviral agents.

16. The use of claim 15, wherein, the tumor is breast cancer, lung cancer, liver cancer, colon cancer, pancreatic cancer, T cell lymphoma, soft tissue sarcoma, Hodgkin's lymphoma, Non-Hodgkin's lymphoma, ovarian cancer or bladder cancer.

17. A use of a gemcitabine amide derivative of the general formula (I) in the preparation of antitumor or antiviral agents, wherein, R is n-butyl.

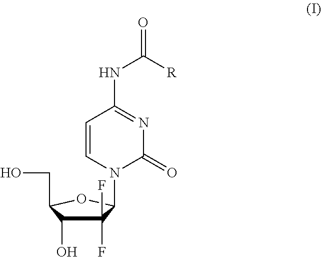

18. The use of claim 17, wherein, the tumor is breast cancer, lung cancer, liver cancer, colon cancer, pancreatic cancer, T cell lymphoma, soft tissue sarcoma, Hodgkin's lymphoma, Non-Hodgkin's lymphoma, ovarian cancer or bladder cancer.

19. A composition, comprising a safe and effective amount of the compound of claim 12, and a pharmaceutically acceptable carrier.

20. The composition of claim 19, wherein, the composition is a tablet, capsule or injection.

21. A composition, comprising a safe and effective amount of a gemcitabine amide derivative of the general formula (I), and a pharmaceutically acceptable carrier, wherein, R is n-butyl.

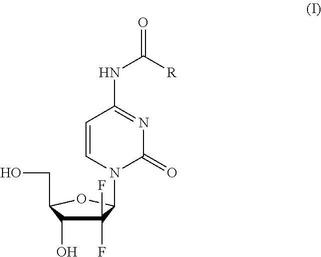

22. The composition of claim 21, wherein, the composition is a tablet, capsule or injection.

23. A preparation method for the compound of claim 12, wherein, said method comprising the following step: the mixture of gemcitabine, anhydrous pyridine, and triethylchlorosilane, was stirred at room temperature; in the meantime, corresponding carboxylic acid was dissolved in acetonitrile, then the condensing agent was added and stirred at room temperature, this solution was added dropwise to the forementioned mixture of gemcitabine, stirred overnight while maintaining at 30-60° C., the reaction mixture was concentrated, and the residue was dissolved in an appropriate amount of an organic solvent, trifluoroacetic acid was added dropwise and stirred to recover the solvent and give the crude product, which was purified by silica gel column chromatography to give the pure target product.

24. A preparation method for the compound of the general formula (I) of claim 12, wherein, said method comprising the following step:
A condensation reaction of gemcitabine with RCOOH in an inert solvent is performed to form the compound of the general formula (I), wherein, R is defined as claim 12.
